(19) United States
(12) Patent Application Publication
(10) Pub. No.: US 2013/0324825 A1
(43) Pub. Date: Dec. 5, 2013

(54) Leadless Pacemaker with Multiple Electrodes

(71) Applicants: Alan Ostroff, Pleasanton, CA (US); Peter M. Jacobson, Livermore, CA (US)

(72) Inventors: Alan Ostroff, Pleasanton, CA (US); Peter M. Jacobson, Livermore, CA (US)

(21) Appl. No.: 13/910,896

(22) Filed: Jun. 5, 2013

Related U.S. Application Data
(60) Provisional application No. 61/655,851, filed on Jun. 5, 2012.

(51) Int. Cl. A61B 5/042; A61N 1/37; A61N 1/365; A61N 1/362

(52) U.S. Cl. CPC A61B 5/042; A61N 1/362; A61N 1/3706; A61N 1/365
USPC 600/374; 607/36; 607/28; 607/17

(57) ABSTRACT

A leadless pacemaker for pacing a heart of a human is provided, which can include any number of features. In some embodiments, the pacemaker can include a hermetic housing, a first electrode configured to fix the pacemaker to the heart, a second electrode exterior to the hermetic housing, a pulse generator disposed in the hermetic housing and configured to generate electrical pulses, the pulse generator being electrically connectable to the first and second electrodes, and a controller disposed in the hermetic housing and operatively connected to the pulse generator and a switching circuit to control the delivery of the electrical pulses between the first electrode or the second electrode and the metallic housing to stimulate the heart. In some embodiments, the pacemaker can include three electrodes, and can pace the heart with a first pair of electrodes and sense the heart with a second pair of electrodes.

### CROSS REFERENCE TO RELATED APPLICATIONS

This application claims the benefit of U.S. Provisional Patent Application No. 61/655,851, filed Jun. 5, 2012, titled “Leadless Pacemaker with Multiple Electrodes”, the contents of which are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Cardiac pacing electrically stimulates the heart when the heart's natural pacemaker and/or conduction system fails to provide synchronized atrial and ventricular contractions at appropriate rates and intervals for a patient's needs. Such bradycardia pacing provides relief from symptoms and even life support for hundreds of thousands of patients. Cardiac pacing may also give electrical overdrive stimulation intended to suppress or convert tachyarrhythmias, again supplying relief from symptoms and preventing or terminating arrhythmias that could lead to sudden cardiac death.

Pacemakers require at least two electrodes to deliver electrical therapy to the heart and to sense the intracardiac electrogram. Traditionally, pacemaker systems are comprised of an implantable pulse generator and lead system. The pulse generators are implanted under the skin and connected to a lead system that is implanted inside the heart with at least one electrode touching the endocardium. The lead system can also be implanted on the epicardial surface of the heart.

Pacemaker lead systems are typically built using a unipolar design, with an electrode at the tip of the lead wire, or bipolar design, with an additional electrode ring often 10 mm proximal to the tip electrode. Additionally, the implanted pulse generator can is often used as a pace/sense electrode. In a conventional pacemaker system, pacing occurs either between the electrode tip and ring, or between the tip and can. Likewise, sensing occurs either between the electrode tip and ring or between the tip and the can.

For evoked potential sensing applications where the evoked response is used as part of an autocapture algorithm, some devices sense the evoked response between the ring and the can when pacing between the tip and the can or when pacing between the tip and the ring. The configurations for pacing and sensing are individually programmable in modern pacemakers.

### SUMMARY OF THE DISCLOSURE

A leadless pacemaker for pacing a heart of a human is provided, comprising a metallic hermetic housing, a first electrode comprising a fixation device exterior to the hermetic housing and configured to affix the pacemaker to the heart, a second electrode exterior to the hermetic housing and supported by an insulating header on or near the hermetic housing, a pulse generator disposed in the hermetic housing and configured to generate electrical pulses, the pulse generator being electrically connectable to the first and second electrodes through two feedthroughs passing through the hermetic housing, and a controller disposed in the hermetic housing and operatively connected to the pulse generator and a switching circuit to control the delivery of the electrical pulses between the first electrode or the second electrode and the metallic housing to stimulate the heart.

In some embodiments, the fixation device is a helical screw.

In another embodiment, the second electrode has a surface area of less than 10 mm².

In some embodiments, the controller is further configured to control the switching circuit to sense electrical activity from the heart between the first electrode and the metallic housing.

In one embodiment, the controller is further configured to control the switching circuit to sense electrical activity from the heart between the second electrode and the metallic housing.

In another embodiment, the controller is further configured to control the switching circuit to sense electrical activity from the heart between the first and second electrodes.

In some embodiments, the controller is further configured to control the switching circuit to sense evoked response between the first electrode and the metallic housing.

In one embodiment, the controller is further configured to control the switching circuit to sense evoked response between the second electrode and the metallic housing.

In another embodiment, the controller is further configured to control the switching circuit to sense evoked response between the first and second electrodes.

In some embodiments, the first electrode is coated with IROX or TiN.

In an additional embodiment, the insulated header is at least 2 mm thick.

Another leadless pacemaker configured to pace a heart of a human is provided, comprising a hermetic housing adapted to be affixed to the heart, a first electrode exterior to the hermetic housing, a second electrode exterior to the hermetic housing, a pulse generator disposed in the hermetic housing and configured to generate electrical pulses, the pulse generator being electrically connectable to the first and second electrodes, and a controller disposed in the hermetic housing and operatively connected to the pulse generator and a switching circuit to control the first electrode as a stimulation electrode and not a sensing electrode and to drive the second electrode as a sensing electrode and not a stimulation electrode.

In one embodiment, the first electrode comprises a header electrode supported by an insulating header on or near the hermetic housing and the second electrode comprises a fixation device adapted to affix the hermetic housing to the heart.

In another embodiment, the first electrode comprises a fixation device adapted to affix the hermetic housing to the heart and the second electrode comprises a header electrode supported by an insulating header on or near the hermetic housing.

In some embodiments, the first electrode is configured to pace the heart.

In another embodiment, the second electrode is configured for evoked response sensing of the heart.

In some embodiments, the hermetic housing comprises a return electrode configured to both pace the heart and sense the heart.

A leadless pacemaker is provided, comprising a hermetic housing comprising a first electrode, a fixation device comprising a second electrode, the fixation device being exterior to the hermetic housing and configured to affix the pacemaker to the heart, a third electrode exterior to the hermetic housing and supported by an insulating header on or near the hermetic housing, a pulse generator disposed in the hermetic housing and configured to generate electrical pulses, the pulse generator being electrically connectable to the first, second, and third electrodes, and a controller disposed in the hermetic housing and operatively connected to the pulse generator and a switching circuit to use a first pair of electrodes chosen from the first, second, and third electrodes for pacing of the heart, and to use a second pair of electrodes chosen from the first, second, and third electrodes for evoked response sensing of the heart.

In some embodiments, the first and second electrodes are used for pacing, and the first and third electrodes are used for sensing.

In other embodiments, the first and second electrodes are used for pacing, and the second and third electrodes are used for sensing.

In additional embodiments, the first and third electrodes are used for pacing, and the first and second electrodes are used for sensing.

In one embodiment, the first and third electrodes are used for pacing, and the second and third electrodes are used for sensing.

A method of treating a heart is provided, comprising affixing a leadless pacemaker to an interior wall of a heart, pacing the heart with a first electrode and a second electrode of the leadless pacemaker, and sensing the heart with a second electrode and a third electrode of the leadless pacemaker.

In some embodiments, the first electrode comprises a fixation device, the second electrode comprises a header electrode supported by an insulating header on or near a hermetic housing of the leadless pacemaker, and the third electrode comprises the hermetic housing.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1A is a pictorial diagram showing an embodiment of a cardiac pacing system that includes a leadless cardiac pacemaker;

FIG. 1B is a schematic block diagram showing interconnection of operating elements of an embodiment of an illustrative leadless cardiac pacemaker;

FIG. 2A is a pictorial diagram showing a leadless pacemaker having three electrodes with an inner fixation electrode and an outer electrode;

FIG. 2B is a pictorial diagram showing a leadless pacemaker showing a leadless pacemaker with an outer fixation electrode and an inner electrode;

FIG. 3 is a schematic showing an exemplary pacing output circuit for a pacemaker having two electrodes;

FIG. 4 is a schematic showing an exemplary sensing circuit for a pacemaker having two electrodes;

FIG. 5 is an exemplary sensing circuit for a pacemaker having three electrodes; and

FIG. 6 is an exemplary output circuit for a pacemaker that can direct pacing to two different electrodes, with respect to a third electrode.

### DETAILED DESCRIPTION

In a leadless pacemaker, a minimum of two electrodes is required, although the details how to achieve high efficiency pacing and sensing electrodes have been overlooked. One of the electrodes is typically referred to as the stimulation electrode and must be close to myocardium. The other electrode, referred to as the return electrode, need not be in direct contact with the myocardium.

To improve the pacing efficiency in a leadless pacemaker and reduce packaging difficulty, the pacemaker's hermetic enclosure may be used as the return electrode. The electrical connection between the stimulation electrode and the internal circuitry (e.g., pulse generator or sensing circuit) must pass through the hermetic enclosure using a hermetic feedthrough. Reducing the number of feedthrough connections can be important in the design of a pacemaker to reduce cost and size, and increase reliability.

With a single pin feedthrough, the stimulation electrode can be integrated onto the fixation element of the leadless pacemaker. For example, a helical screw can provide mechanical support in addition to acting as the stimulation electrode. As an alternative, a stimulation electrode other than the helical attachment element can be connected to the internal pulse generation circuitry through the single pin feedthrough.

For sensing, a large micro surface area is important to reduce the source impedance seen by the pacemaker's input amplifier. This is typically accomplished by coating the electrode used for sensing by IROX or TiN.

In a leadless pacemaker with a single pin feedthrough, the sense amplifier can connect between the stimulation electrode and the return electrode. With a single pin configuration, the distance between the stimulation electrode and the can for optimal sensing can be at least 2 mm in the atrium and ventricle. Less than 2 mm spacing results in poor sensing performance. The minimum 2 mm spacing can be accomplished using an insulated header between the stimulation electrode and the return electrode, or additional insulation can be added to the hermetic enclosure to increase the distance between the stimulation electrode and the return electrode. Materials such as parylene, silicone or ePTFE for example could be used as an insulator surrounding the hermetic enclosure. Typically an electrode distance of 10 mm is desirable between the stimulation electrode and the return electrode.

However, in a leadless pacemaker with at least two feedthrough pins, the sensing configuration can be selected among one of three possible pairs of electrodes: stimulation electrode #1 to housing, stimulation electrode #2 to housing, or stimulation electrode #1 to stimulation electrode #2. The advantage is that at least one electrode will not be used for pacing and therefore acts as an indifferent electrode. This indifferent electrode provides the ability to sense the evoked response more easily because the electrode/tissue interface has not been disturbed by stimulation pulse required for pacing.

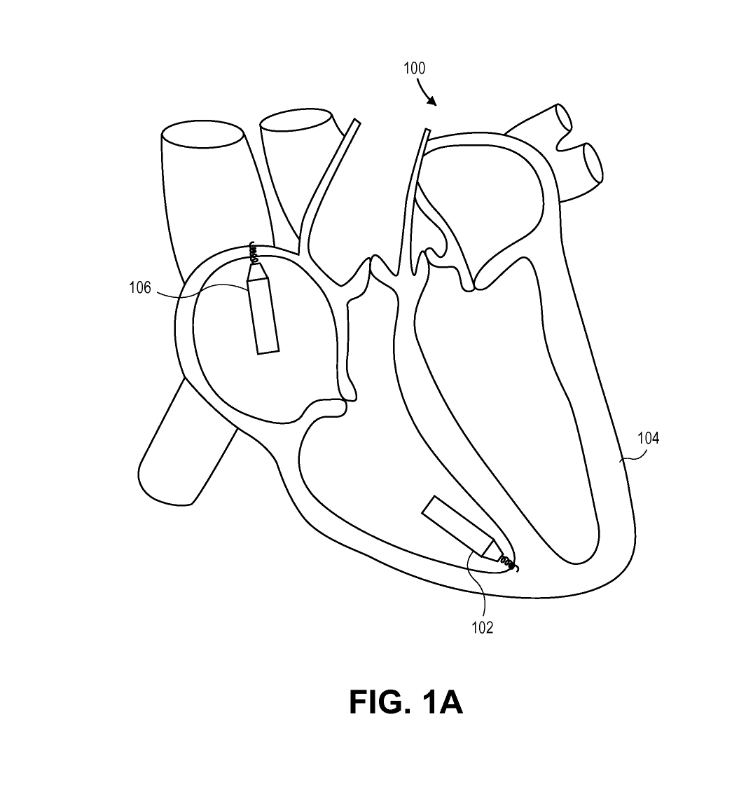

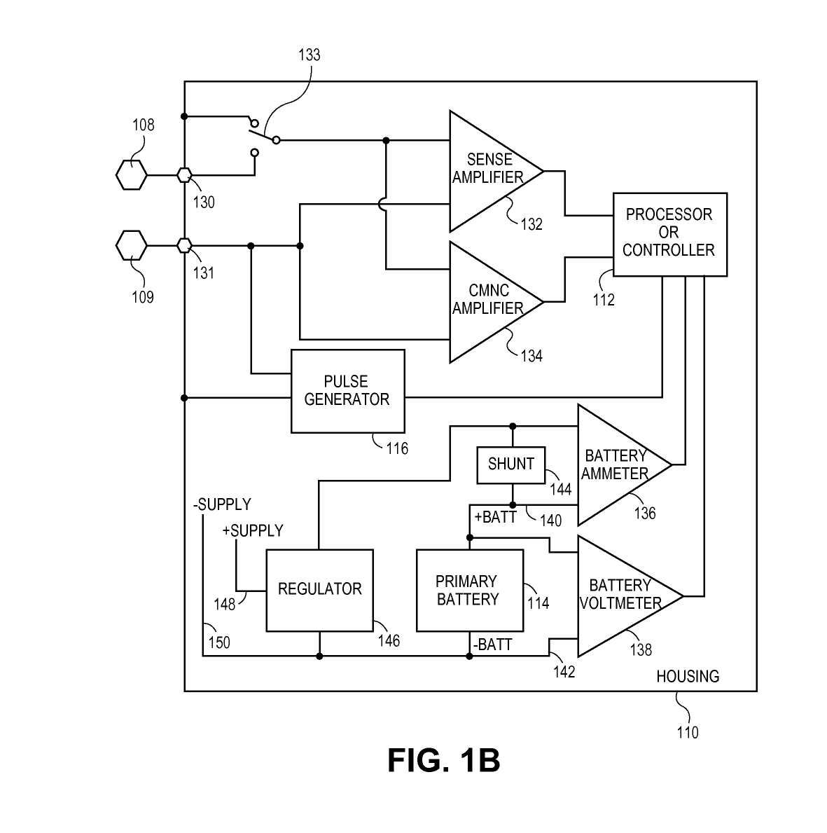

FIG. 1A shows two leadless cardiac pacemakers 102 and 106 attached to the cardiac wall 104 of the heart 100.

FIG. 1B shows a schematic diagram of the pacemakers of FIG. 1A. In one embodiment, the leadless cardiac pacemaker 102 (or 106) can comprise a metallic hermetic housing 110 and multiple electrodes 108 and 109 coupled to the housing 110, i.e. within, on, or near the housing 110. The metallic hermetic housing 110 can be configured as a return electrode, as discussed further below. A switch 133 can be used to selectively connect the signal sensing circuitry to housing 110 or electrode 108 for this purpose. Hermetic feedthroughs 130, 131 permit the electrodes 108 and 109 to electrically connect with components inside the housing 110.

A pulse generator 116 can be located inside the housing 110 and electrically coupled to the electrode 109 and the hermetic housing 110. The pulse generator 116 can be configured for sourcing energy internal to the housing 110 and generating and delivering electrical pulses to the electrode 109 and the hermetic housing 110. This delivery of energy can cause cardiac contractions to pace the heart. In some embodiments, the pulse generator 116 also conveys information or communication signals to one or more devices 106 (see FIG. 1A) external to the pacemaker 102, such as another pacemaker or an external programmer.

A processor 112 can also be hermetically contained within the housing 110 and can be communicatively coupled to the electrodes 108 and the hermetic housing 110. The processor 112 can sense electrical activity from the muscle of the cardiac chamber through the electrodes 108 and/or the hermetic housing 110. The processor 112 can further control electrical pulse delivery at least partly based on the sensed activity.

The housing 110 can also contain circuits 132 for sensing cardiac activity from the electrodes 108 and 109 or alternatively from electrodes 108 and housing 110. In some embodiments, circuits 134 for receiving information from at least one other device via the same electrodes as those used to sense cardiac activity. In some embodiments, the pacemaker 102 further contains circuits for monitoring device health, for example a battery current monitor 136 and a battery voltage monitor 138. The processor 112 is configured to control these operations in a predetermined manner. In the case wherein the housing acts as a return electrode, the circuits would also be electrically coupled to the housing via switch 133. The circuits 132, 134 can be configured to amplify signals received from the electrode 108 and to detect cardiac contractions, and further can receive information from an external device or devices, such as pacemaker 106. In other embodiments, an additional amplifier could be added to the circuit so that, e.g., frequency response and gain can be optimized for sending evoked potential.

The housing 110 further contains a primary battery 114 to provide power for pacing, sensing, and/or communication. The primary battery 114 can have positive terminal 140 and negative terminal 142.

In some embodiments, current from the positive terminal 140 of primary battery 114 flows through a shunt 144 to a regulator circuit 146 to create a positive voltage supply 148 suitable for powering the remaining circuitry of the pacemaker 102. The shunt 144 enables the battery current monitor 136 to provide the processor 112 with an indication of battery current drain and indirectly of device health.

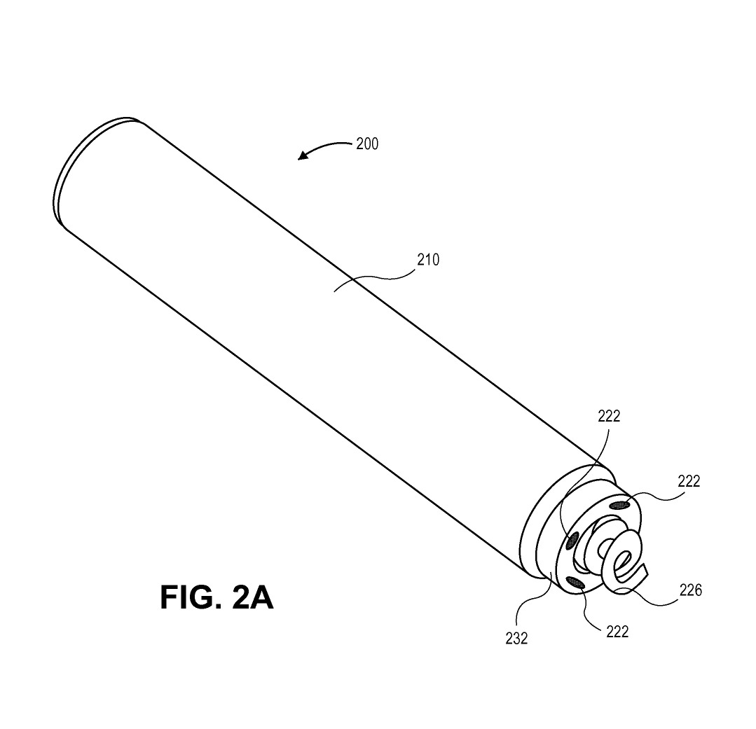

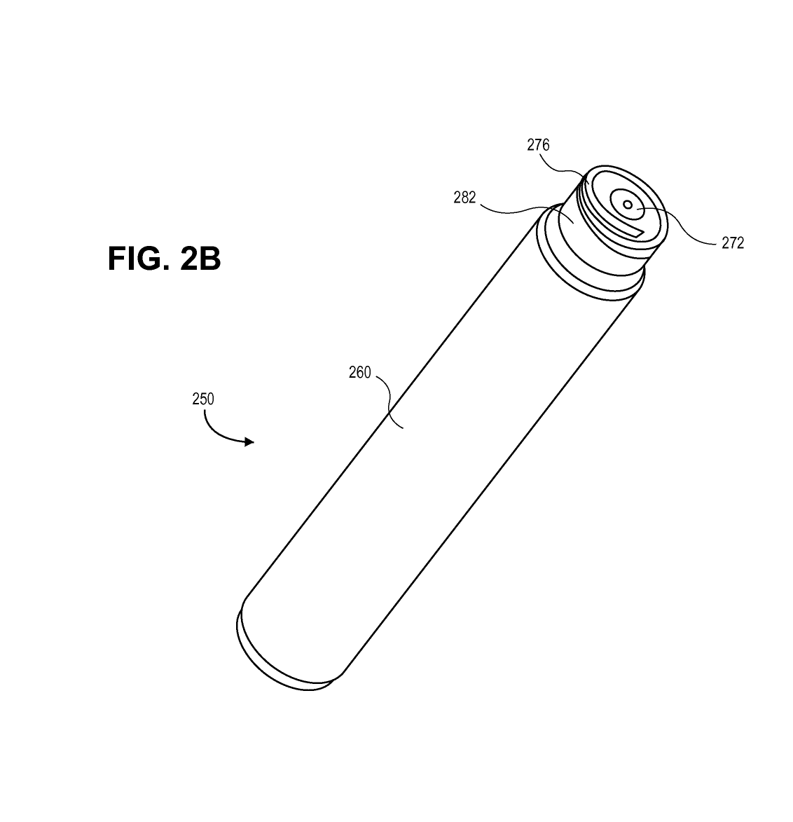

FIGS. 2A and 2B show leadless pacemakers with bipolar electrode designs using the hermetic housing as a return electrode for sensing (including evoked response sensing) and pacing. That is, the leadless pacemaker can include two electrodes in addition to the use of the hermetic housing as the return electrode (for a total of three electrodes). The first electrode can be used for pacing and not for sensing, the second electrode can be used for sensing and not for pacing, and the third electrode (i.e., the housing) can be used as the return electrode for pacing and sensing. Other combinations of electrodes are possible, of course, for the purposes of pacing and sensing.

As shown in FIG. 2A, a leadless pacemaker 200 includes a metallic hermetic housing 210, an insulating header 232 having four header electrodes 222 (of which three are visible in FIG. 2A), and a helical fixation device 226. The metallic hermetic housing 210 can be composed of titanium (such as grade 1 titanium), stainless steel, or another biocompatible metallic alloy.

The fixation device 226 can be configured to provide mechanical support for the pacemaker 200, i.e., to affix the pacemaker 200 to the heart. The fixation device 226 can be further configured to act as either a stimulation electrode or a sensing electrode and therefore be in electrical contact with the pulse delivery system and controller located inside the hermetic housing 210. The fixation device 226 can pass through the housing 210 using a dual pin feedthrough (not shown). Although the fixation device 226 is shown in a helical screw configuration, other configurations are possible, such as a harpoon configuration. The fixation device 226 can be comprised of a low polarization material such as TiN or IROX, e.g., can be coated with the low polarization material. Using IROX or TiN can advantageously provide a large micro surface area to reduce source impedance seen by the pacemaker's amplifier, particularly for the electrode used for sensing.

The header electrodes 222 can be electrically coupled and can be configured to act together either as a stimulation electrode or a sensing electrode and are therefore in electrical contact with the pulse delivery system and controller located inside the hermetic housing 210. Although the header electrodes 222 are shown as multiple separate (but electrically coupled) electrodes, it could also be a single annular electrode, a hemispherical “bump” electrode or any other electrode configuration. The header electrodes 222 can be comprised of, or coated with, a low polarization material such as TiN or IROX. In some embodiments, the header electrodes 222 can have an area of less than 10 mm².

Referring still to FIG. 2A, the header electrodes 222 can act as a stimulation electrode or a sensing electrode while the fixation device 226 can act as the opposite (e.g., the fixation device can act as the sensing electrode if the header electrodes act as the stimulation electrode, and vice versa). Thus, for example, the header electrode 222 can act as a stimulation electrode and not as a sensing electrode while the fixation device 226 can act as a sensing electrode and not a stimulation electrode. The hermetic housing 210 can act as a return electrode for both pacing and stimulation. In some embodiments, the hermetic housing 210 can have a surface area of greater than ten times the surface area of the header electrodes 222 or the fixation device 226. Having a surface area of greater than ten times the surface area of the stimulation electrode advantageously increases the pacing efficiency. While the stimulation electrode, i.e., the fixation device 226 or alternatively the header electrodes 222, should be in contact with the myocardium for proper stimulation, the return electrode, i.e., the housing metallic housing, need not be contact with the myocardium. In other embodiments, the header electrode can be used as a stimulation electrode and as a sensing electrode, while the fixation electrode can be used for evoked potential sensing.

FIG. 2B shows an alternative embodiment in which the leadless pacemaker 250 has a metallic hermetic housing 260, an insulating header 282, a central electrode 272, and a helical fixation device 276. As before, the metallic hermetic housing 260 can be composed of titanium (such as grade 1 titanium), stainless steel, or another biocompatible metallic alloy.

As in the other embodiment, the fixation device 276 is configured to provide mechanical support for the pacemaker 250, i.e., to fix the pacemaker 250 to the heart. The fixation device 276 is further configured to act as either a stimulation electrode or a sensing electrode and is therefore in electrical contact with the pulse delivery system and controller located inside the hermetic housing 260. The fixation device 276 passes through the housing 260 using a dual pin feedthrough (not shown). Although the fixation device 276 is shown in a helical screw configuration, other configurations are possible, such as a harpoon configuration. The fixation device 276 can be comprised of a low polarization material such as TiN or IROX, e.g., can be coated with the low polarization material. Using IROX or TiN can advantageously provide a large micro surface area to reduce source impedance seen by the pacemaker's amplifier, particularly for the electrode used for sensing.

The central electrode 272 can be configured to act either as a stimulation electrode or a sensing electrode and is therefore in electrical contact with the pulse delivery system and controller located inside the hermetic housing 260. The central electrode 272 can be comprised of, or coated with, a low polarization material such as TiN or IROX and can have an area of less than 10 mm².

Referring still to FIG. 2B, the central electrode 272 can act as a stimulation electrode or a sensing electrode while the fixation device 276 can act as the opposite. Thus, for example, the header electrode 272 can act as a stimulation electrode and not as a sensing electrode while the fixation device 276 can act as a sensing electrode and not a stimulation electrode. The hermetic housing 260 can act as a return electrode for both pacing and stimulation. The hermetic housing 260 can have a surface area of greater than ten times the surface area of the central electrode 272 or the fixation device 276.

In some embodiments, there is a space of at least 2 mm, such as approximately 10 mm, between the sensing electrode, i.e., the fixation element or the header electrode, and the metallic housing serving as the return electrode. Having a space of 2 mm or more, such as approximately 10 mm, ensures better sensing performance as a result of the speed of the propagating depolarization gradient. The space can be accomplished using an insulated header between the stimulation and the return electrode or adding additional insulation to the hermetic housing to increase the distance between the sensing electrode and the return electrode. Materials such as parylene, silicone, or ePTFE can be used as the insulator.

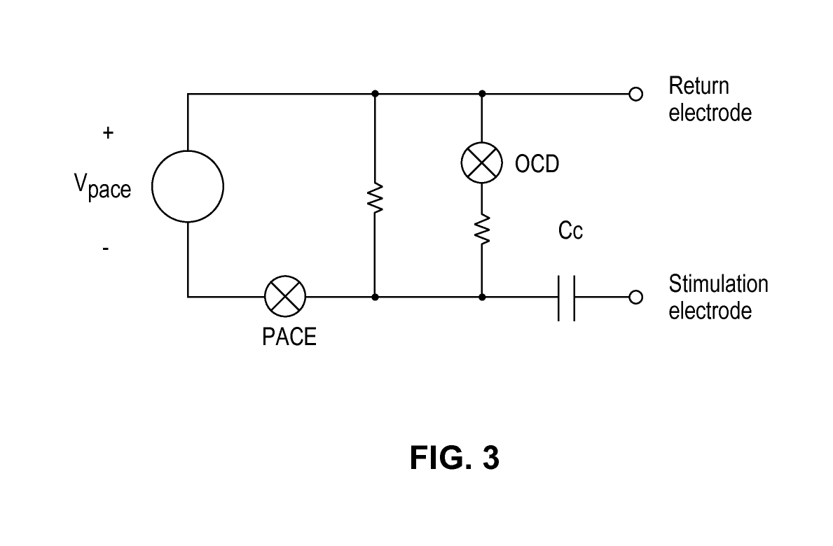

An exemplary pacing output circuit is shown in FIG. 3. In the circuit of FIG. 3, the pacing voltage connects to the return and stimulation electrodes through a coupling capacitor Cc when the PACE switch is asserted. In one embodiment, the PACE switch is asserted for 0.4 ms every pacing cycle. Following pacing, the OCD switch is closed. In one embodiment, the OCD switch is closed for approximately 20 ms to discharge the coupling capacitor Cc and provide charge balancing.

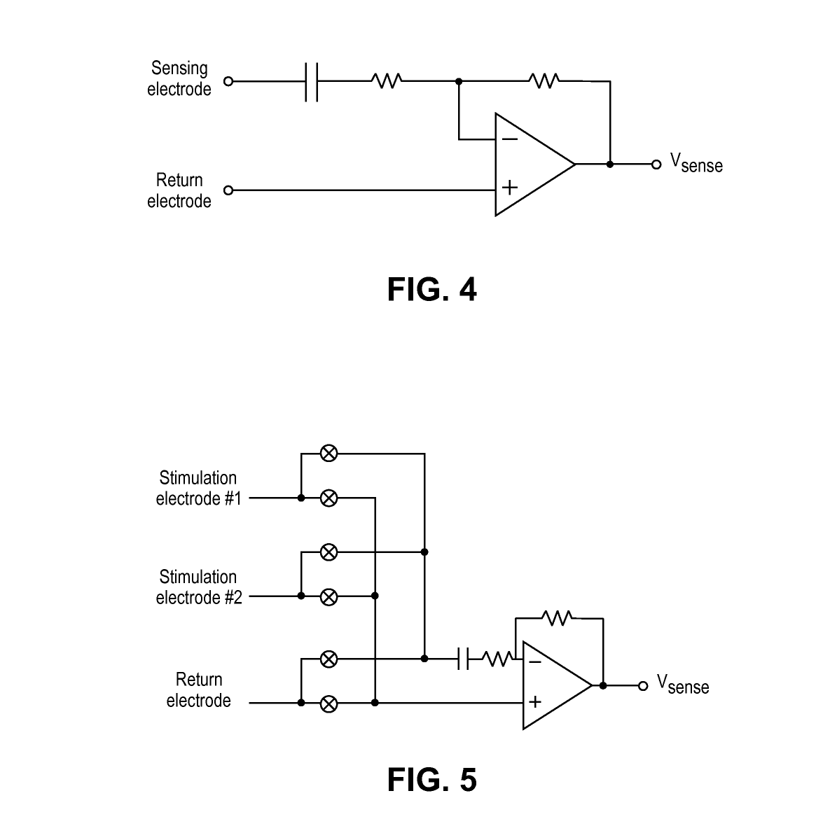

An exemplary sensory input circuit is shown in FIG. 4. In the circuit of FIG. 4, a sense amplifier connects between the sensing electrode and the return electrode. There are many other ways to implement a sense amplifier, as known to those skilled in the art. The circuit of FIG. 4 simply shows that there are at least two connections that can be connected to any combination of two electrodes. The circuit for normal cardiac sensing and the circuit for evoked potential sensing may use the same circuit, but that is not a requirement. There may be a separate amplifier for each purpose that is fined tuned in terms of gain and bandwidth for optimal sensing for each purpose.

An exemplary output circuit for a pacemaker having separate stimulation and sensing electrodes, as described with respect to FIGS. 2A and 2B, is shown in FIG. 5. As shown in FIG. 5, an electrical multiplexer can allow the sensing pair to be determined by the pacemaker's controller.

Although the embodiments above suggest that the hermetic housing 110 is the return electrode for both pacing and sensing, it need not be. Indeed, in some embodiments, the header electrode or the fixation device can act as a return electrode. Further, the sensing and/or pacing can occur between any combination of electrode pairs. For example, pacing could be performed between the fixation device and the header electrode, between the header electrode and the hermetic housing, and between the hermetic housing and the fixation device. A different pair can then be used for sensing.

Further, in some embodiments, pairs of electrodes can be used for normal sensing separate from those for evoked response sensing. For example, the pacemaker could include a second multiplexor and high-pass filter capacitor. By including a second multiplexer and high-pass filter capacitor, the sensing amplifier could be configured to sense any electrode pair for normal sensing and independently select any electrode pair for evoked response sensing.

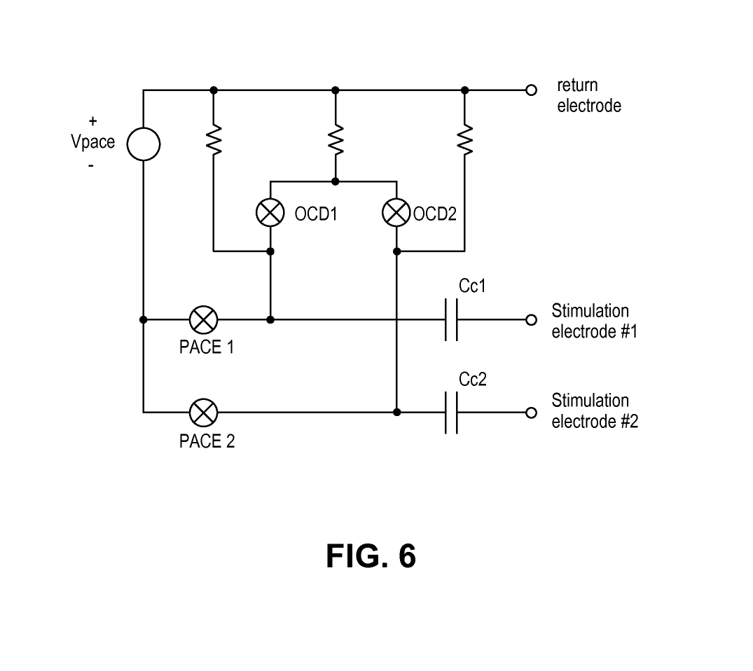

Further, referring to FIG. 6, in some embodiments, the fixation device and the header can each be configured as stimulation electrodes with the housing serving as the return electrode. In this embodiment, the pacemaker can be configured to electrically switch between either stimulation electrode if one were to suddenly lose the ability to stimulate the heart, i.e. suffer from exit block. As shown in FIG. 6, the pacing output circuit can direct pacing to either stimulation electrode depending on whether SW1 or SW2 is asserted.

Advantageously, using both the fixation device 226 and the housing 210 of FIG. 2A as electrodes reduces the size and weight of the pacemaker. Further, having a separate stimulation electrode and pacing electrode advantageously allows the evoked response to be sensed more easily because the electrode and/or tissue interface has not been disturbed by the stimulation pulses required for pacing. Having a limited number of feedthroughs in a pacemaker, such as two feedthroughs as described herein, also advantageously reduces cost and size, and increases reliability.

Modifications to the invention will be apparent to those skilled in the art. For example, in some embodiments, a steroid can be included on or near the stimulation electrodes to reduce fibrosis and improve pacing thresholds.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A leadless pacemaker for pacing a heart of a human comprising:
a metallic hermetic housing;
a first electrode comprising a fixation device exterior to the hermetic housing and configured to affix the pacemaker to the heart;
a second electrode exterior to the hermetic housing and supported by an insulating header on or near the hermetic housing;
a pulse generator disposed in the hermetic housing and configured to generate electrical pulses, the pulse generator being electrically connectable to the first and second electrodes through two feedthroughs passing through the hermetic housing; and
a controller disposed in the hermetic housing and operatively connected to the pulse generator and a switching circuit to control the delivery of the electrical pulses between the first electrode or the second electrode and the metallic housing to stimulate the heart.

2. The leadless pacemaker of claim 1 wherein the fixation device is a helical screw.

3. The leadless pacemaker of claim 1 wherein the second electrode has a surface area of less than 10 mm².

4. The leadless pacemaker of claim 1 wherein the controller is further configured to control the switching circuit to sense electrical activity from the heart between the first electrode and the metallic housing.

5. The leadless pacemaker of claim 1 wherein the controller is further configured to control the switching circuit to sense electrical activity from the heart between the second electrode and the metallic housing.

6. The leadless pacemaker of claim 1 wherein the controller is further configured to control the switching circuit to sense electrical activity from the heart between the first and second electrodes.

7. The leadless pacemaker of claim 1 wherein the controller is further configured to control the switching circuit to sense evoked response between the first electrode and the metallic housing.

8. The leadless pacemaker of claim 1 wherein the controller is further configured to control the switching circuit to sense evoked response between the second electrode and the metallic housing.

9. The leadless pacemaker of claim 1 wherein the controller is further configured to control the switching circuit to sense evoked response between the first and second electrodes.

10. The leadless pacemaker of claim 1 wherein the first electrode is coated with IROX or TiN.

11. The leadless pacemaker of claim 1 wherein the insulated header is at least 2 mm thick.

12. A leadless pacemaker configured to pace a heart of a human, comprising:
a hermetic housing adapted to be affixed to the heart;
a first electrode exterior to the hermetic housing;
a second electrode exterior to the hermetic housing;
a pulse generator disposed in the hermetic housing and configured to generate electrical pulses, the pulse generator being electrically connectable to the first and second electrodes; and
a controller disposed in the hermetic housing and operatively connected to the pulse generator and a switching circuit to control the first electrode as a stimulation electrode and not a sensing electrode and to drive the second electrode as a sensing electrode and not a stimulation electrode.

13. The leadless pacemaker of claim 12 wherein the first electrode comprises a header electrode supported by an insulating header on or near the hermetic housing and the second electrode comprises a fixation device adapted to affix the hermetic housing to the heart.

14. The leadless pacemaker of claim 12 wherein the first electrode comprises a fixation device adapted to affix the hermetic housing to the heart and the second electrode comprises a header electrode supported by an insulating header on or near the hermetic housing.

15. The leadless pacemaker of claim 12 wherein the first electrode is configured to pace the heart.

16. The leadless pacemaker of claim 15 wherein the second electrode is configured for evoked response sensing of the heart.

17. The leadless pacemaker of claim 16 wherein the hermetic housing comprises a return electrode configured to both pace the heart and sense the heart.

18. A leadless pacemaker, comprising:
a hermetic housing comprising a first electrode;
a fixation device comprising a second electrode, the fixation device being exterior to the hermetic housing and configured to affix the pacemaker to the heart;
a third electrode exterior to the hermetic housing and supported by an insulating header on or near the hermetic housing;
a pulse generator disposed in the hermetic housing and configured to generate electrical pulses, the pulse generator being electrically connectable to the first, second, and third electrodes; and
a controller disposed in the hermetic housing and operatively connected to the pulse generator and a switching circuit to use a first pair of electrodes chosen from the first, second, and third electrodes for pacing of the heart, and to use a second pair of electrodes chosen from the first, second, and third electrodes for evoked response sensing of the heart.

19. The leadless pacemaker of claim 18 wherein the first and second electrodes are used for pacing, and the first and third electrodes are used for sensing.

20. The leadless pacemaker of claim 18 wherein the first and second electrodes are used for pacing, and the second and third electrodes are used for sensing.

21. The leadless pacemaker of claim 18 wherein the first and third electrodes are used for pacing, and the first and second electrodes are used for sensing.

22. The leadless pacemaker of claim 18 wherein the first and third electrodes are used for pacing, and the second and third electrodes are used for sensing.

23. A method of treating a heart, comprising:
affixing a leadless pacemaker to an interior wall of a heart;
pacing the heart with a first electrode and a second electrode of the leadless pacemaker; and
sensing the heart with a second electrode and a third electrode of the leadless pacemaker.

24. The method of claim 23 wherein the first electrode comprises a fixation device, the second electrode comprises a header electrode supported by an insulating header on or near a hermetic housing of the leadless pacemaker, and the third electrode comprises the hermetic housing.
